(19) United States
(12) Patent Application Publication
(10) Pub. No.: US 2026/0076598 A1
(43) Pub. Date: Mar. 19, 2026

(54) METHODS, SYSTEMS, AND APPARATUSES FOR COLLECTING A HIGH VOLUME OF BODILY FLUID USING HANDHELD BODILY FLUID COLLECTION DEVICES

(71) Applicant: Tasso, Inc., Seattle, WA (US)

(72) Inventors: Sebastian Crisologo Bibat, Seattle, WA (US); Bryce Petersen, Seattle, WA (US); Yuri Miyamoto, Seattle, WA (US); Erwin Berthier, Seattle, WA (US); Jared Andrew Salstrom, Lake Stevens, WA (US); Michael Lin, Seattle, WA (US)

(21) Appl. No.: 19/331,907

(22) Filed: Sep. 17, 2025

Related U.S. Application Data
(60) Provisional application No. 63/695,620, filed on Sep. 17, 2024.

(51) Int. Cl. A61B 5/151; A61B 5/15

(52) U.S. Cl. CPC A61B 5/15105; A61B 5/150099; A61B 5/150267

(57) ABSTRACT

Methods, systems, and apparatuses for collecting a high volume of bodily fluid using a handheld bodily fluid collection device are disclosed. A handheld bodily fluid collection device includes a housing having an opening, a skin-piercing assembly positioned at least partially within the housing, and an actuator coupled to the skin-piercing assembly. The actuator can be actuated to cause a blade of the skin-piercing assembly to form an incision in the skin of a patient. In some embodiments, a vacuum mechanism configured to dynamically generate a vacuum is applied to the patient's skin to facilitate collection of the bodily fluid. The handheld bodily fluid collection device includes a surface that contacts the skin of the subject and is shaped to define an accommodating space into which a portion of the subject's skin is drawn (e.g., via vacuum pressure).

### CROSS-REFERENCE TO RELATED APPLICATION

This application claims the benefit of U.S. Provisional Patent Application No. 63/695,620, filed Sep. 17, 2024, and titled “METHODS, SYSTEMS, AND APPARATUSES FOR COLLECTING A HIGH VOLUME OF BODILY FLUID USING HANDHELD BODILY FLUID COLLECTION DEVICES,” which is incorporated herein by reference in its entirety.

### TECHNICAL FIELD

The present technology is related to collecting bodily fluid from a subject and, in particular, to methods, systems, and apparatuses for increasing the collection volume of bodily fluids via handheld bodily fluid collection devices.

### BACKGROUND

Devices, systems, and methods to collect bodily fluids, such as blood, are widely used in personalized, clinical, and field medical applications. Biological samples are commonly collected using simple lancing devices or more sophisticated devices that require trained personnel (e.g., phlebotomy venipunctures). Transferring bodily fluids to a container, receptacle, or analysis device often requires several steps, which can be time-consuming, prone to error, and/or cumbersome. Moreover, collecting a sufficient volume of bodily fluids can be difficult, particularly for untrained users employing many existing personalized devices.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1A is a perspective view of a bodily fluid collection device configured in accordance with embodiments of the present technology.

FIG. 1B is a perspective view of the bodily fluid collection device of FIG. 1A during use in accordance with embodiments of the present technology.

FIG. 1C is a perspective view illustrating detachment of a collection reservoir from the bodily fluid collection device of FIGS. 1A and 1B in accordance with embodiments of the present technology.

FIG. 2 is a partially schematic cross-sectional side view of a bodily fluid collection device configured in accordance with additional embodiments of the present technology.

FIG. 3 is a partially schematic cross-sectional side view of a bodily fluid collection device configured in accordance with further embodiments of the present technology.

FIGS. 4A and 4B are partially schematic cross-sectional perspective views of a bodily fluid collection device configured in accordance with embodiments of the present technology.

FIG. 5 is a partially schematic cross-sectional side view of a bodily fluid collection device configured in accordance with further embodiments of the present technology.

FIG. 6A is a perspective view of a bodily fluid collection device with a temperature/vibration accessory device configured in accordance with embodiments of the present technology.

FIG. 6B is a perspective view of a bodily fluid collection device with a temperature/vibration accessory device configured in accordance with embodiments of the present technology.

FIG. 6C is a perspective view of a bodily fluid collection device with a temperature/vibration accessory device configured in accordance with further embodiments of the present technology.

FIG. 6D is a partially cross-sectional side view of the bodily fluid collection device with the temperature/vibration accessory device of FIG. 6C.

FIG. 7A is a perspective view of a bodily fluid collection device with a pressure accessory device configured in accordance with embodiments of the present technology.

FIG. 7B is a perspective view of a bodily fluid collection device with a pressure accessory device configured in accordance with additional embodiments of the present technology.

FIG. 8 is a display diagram and table based on data generated by the inventors in accordance with embodiments of the present technology.

FIGS. 9A and 9B are display diagrams and tables based on data generated by the inventors in accordance with additional embodiments of the present technology.

### DETAILED DESCRIPTION

The present technology is directed generally to devices and methods for collecting a high volume of bodily fluid, such as blood, from a subject. A handheld bodily fluid collection device configured in accordance with the present technology can include a housing having an opening, a skin-piercing assembly positioned at least partially within the housing, and an actuator coupled to the skin-piercing assembly. The actuator can be actuated to cause a blade of the skin-piercing assembly to form an incision in the skin of a patient. In some embodiments, the devices and methods disclosed herein use a vacuum mechanism configured to dynamically generate a vacuum that is applied to the patient's skin to facilitate the collection of the bodily fluid. In some embodiments, the handheld bodily fluid collection devices disclosed herein include a surface that contacts the skin of the subject and is shaped to define an accommodating space into which a portion of the subject's skin is drawn (e.g., via vacuum pressure). In some embodiments, the surface is a rigid dome-like structure (e.g., a concave dome with respect to the housing). In some embodiments, the surface is flat or substantially flat with respect to a plane of the subject's skin (e.g., a plane of the surface is approximately parallel to the skin).

In some embodiments, devices configured in accordance with the present technology include one or more temperature/vibration accessories configured to apply heat and/or vibrations to an area proximate the incision site. In some embodiments, heat and/or vibrations are applied directly to the incision site. The temperature/vibration accessory can be a separate component from the bodily fluid collection device. In other embodiments, however, the temperature/vibration accessory is integral with the bodily fluid collection device.

In some embodiments, the present technology includes one or more pressure accessories configured to apply pressure to an area of a subject's skin to increase blood flow through the incision site. In some embodiments, pressure is applied directly to the incision site. In other embodiments, however, pressure is applied to an area of the skin that does not include the incision site.

In some embodiments, vacuum pressure is applied by the bodily fluid collection device in an oscillating pattern configured to increase blood flow through the incision site. Vacuum pressure is oscillated automatically via an actuator motor. In some embodiments, vacuum pressure is oscillated manually (e.g., a user manually manipulates one or more valves).

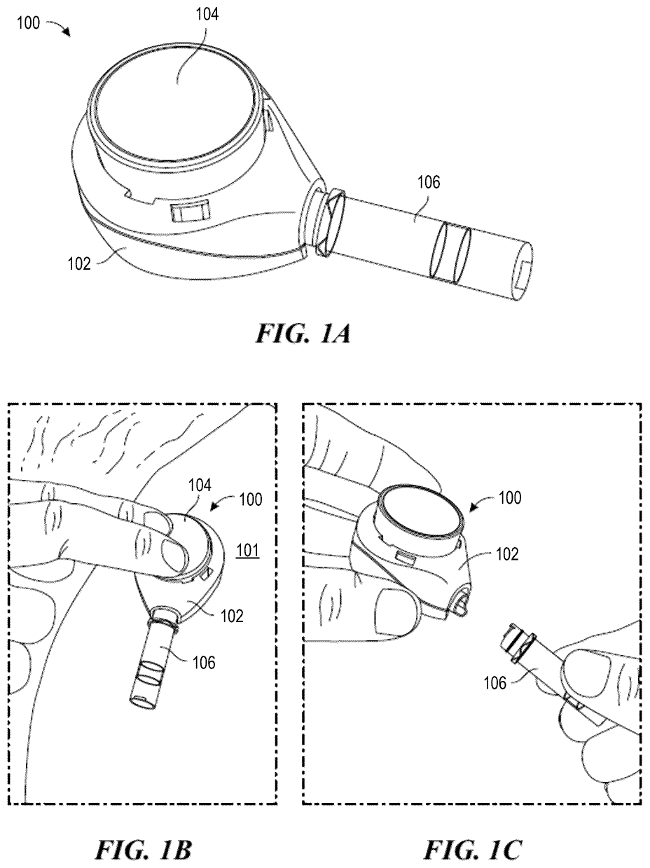

Specific details of several embodiments of the present technology are described herein with reference to FIGS. 1A-6B. However, the present technology may be practiced without some of these specific details. In some instances, well-known structures and techniques often associated with bodily fluid collection devices, improving bodily fluid collection volume, and the like, have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements may be arbitrarily enlarged to improve legibility. Component details may be abstracted in the Figures to exclude details such as the position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

The headings provided herein are for convenience only and should not be construed as limiting the subject matter disclosed.

### A. Select Embodiments of Bodily Fluid Collection Devices

FIG. 1A is a perspective view of a bodily fluid collection device 100 (“device 100”) configured in accordance with embodiments of the present technology. The device 100 can be handheld with a size that is easily grasped and manipulated by one or both of a patient's hands. Such handheld devices advantageously allow a patient to collect a bodily fluid sample (e.g., a blood sample) without assistance from another individual. In some embodiments, the handheld devices of the present technology can be operated by a layperson outside of a medical setting (e.g., at home or in a field clinic) and without the aid of a medical professional.

In the illustrated embodiment, the device 100 includes a housing 102 and an actuator 104. The actuator 104 (e.g., a button) can be movable relative to the housing 102 to actuate/initiate withdrawal of a bodily fluid (e.g., blood) from the patient. The housing 102 is removably coupled to a collection reservoir 106 (e.g., a tube, a collection device, a transfer device, a cartridge, and/or the like) for receiving the bodily fluid withdrawn from the patient. The collection reservoir 106 can act as a removable and standardized container for bodily fluid that can be detached and used in clinical and laboratory equipment or workflows (e.g., for diagnostics and/or biomarker detection).

FIG. 1B is a perspective view of the bodily fluid collection device 100 of FIG. 1A in use by the patient in accordance with embodiments of the present technology. To collect a bodily fluid sample, the device 100 is applied to a patient's body, with a bottom surface of the housing 102 positioned against the skin 101 of the patient and the actuator 104 positioned away from the skin 101. Actuating (e.g., pressing, twisting, pulling) the actuator 104 deploys a skin-piercing feature (e.g., a blade, lancet) from within the device 100 to pierce the skin 101. Subsequent retraction of the actuator 104 away from the skin generates a vacuum within the device 100 that acts against the patient's skin either directly or indirectly, and before and/or after deployment of the skin-piercing feature. Bodily fluid from the resulting incision is withdrawn into the housing 102 and collected into the collection reservoir 106.

FIG. 1C is a perspective view illustrating detachment of the collection reservoir 106 from the bodily fluid collection device 100 of FIGS. 1A and 1B in accordance with embodiments of the present technology. Once a desired amount of bodily fluid has been collected into the collection reservoir 106, the device 100 is removed from the skin 101, and the collection reservoir 106 is detached from the housing 102. In some embodiments, the device 100 can include one or more features that are generally similar to or identical to the bodily fluid collection devices disclosed in (i) U.S. patent application Ser. No. 17/006,248, titled “BODILY FLUID COLLECTION DEVICES AND RELATED METHODS,” and filed Aug. 28, 2020; and/or (ii) U.S. Pat. No. 11,633,136, titled “BODILY FLUID COLLECTION DEVICES AND RELATED METHODS,” and filed Feb. 25, 2022, each of which is incorporated herein by reference in its entirety.

### B. Select Embodiments of Collecting a High Volume of Bodily Fluid Using Handheld Bodily Fluid Collection Devices

Typically, blood for blood tests is withdrawn from a patient by collecting blood directly from a vein of the patient (e.g., via a venipuncture). However, blood for blood tests often needs to be withdrawn outside of a medical setting and without the aid of a medical professional. Personalized, handheld blood collection devices can be used to obtain such blood, but there can be challenges with collecting a sufficient volume of blood using such devices, particularly when employed by laypersons. To make blood collection accessible to a wide variety of people, bodily fluid collection devices (e.g., device 100 of FIGS. 1A-1C) can employ various technologies that simplify blood collection and increase the volume of blood collected.

For example, as disclosed herein, bodily fluid collection devices can employ vacuum pressure to help draw blood from the incision site into the collection device (e.g., collection reservoir 106). In particular, the vacuum pressure can increase capillary action within the skin and increase the amount of blood withdrawn/collected. Thus, as demonstrated in the embodiments disclosed herein, bodily fluid collection devices can be configured to engage additional skin area at and/or adjacent to the incision size, and thereby optimize blood volume collection.

The portion of the skin exposed to vacuum pressure is drawn into an accommodating space of the device. In some embodiments, the shape and area of the accommodating space can be defined by the shape and area of an internal cavity of the device housing. In some embodiments, the shape and area of the accommodating space can be defined by the shape of a lower surface of the housing configured to interface/contact the skin.

The portion of the skin in the accommodating space is pierced by a piercing element (e.g., a blade, a needle, etc.) positioned in the housing of the bodily fluid collection device. The piercing element moves through an opening to pierce the skin. Blood is drawn through the opening (e.g., via vacuum pressure and/or fluidic channels) and collected by a collection cartridge. In some embodiments, the bodily fluid collection device includes a single opening through which skin is drawn by vacuum and through which a piercing element moves to pierce the skin (e.g., the lower portion/surface of a collection device housing defines one opening into which the subject's skin is drawn and where the skin is pierced). In some embodiments, the bodily fluid collection device includes multiple smaller openings configured in a mesh pattern to increase the surface area of skin exposed to vacuum while maintaining a lower surface boundary configured to interface/contact the skin. Maintaining such a boundary can limit bruising due to stretching and/or doming of the skin into the accommodating space.

As a further example, oscillating vacuum pressure over the course of the blood collection procedure is expected to increase blood volume yield. For example, by first applying vacuum pressure at and/or adjacent to an incision site (e.g., during an initial stage of blood collection), and then reducing vacuum pressure to atmospheric pressure, blood is allowed to recirculate into the capillaries drained during the initial stages of blood collection. Vacuum pressure can be applied again once blood has been recirculated, thereby increasing the overall volume of blood obtained. Vacuum pressure can be oscillated manually (e.g., via a user manipulating valves) or automatically via an electric motor.

Additionally, application of various stimuli, such as heat, vibrations, and/or pressure, to an incision site and/or to an area of skin adjacent to the incision site is expected to increase the volume of blood collected. For example, applying heat to skin adjacent to an incision site dilates capillaries and increases local blood flow. Physical vibrations stimulate tissue, causing local blood flow to increase. Further, applying pressure (positive or negative) to a larger area of skin, including the bodily fluid collection device, or at targeted sites adjacent to the collection device, can further increase local blood flow. As described in greater detail below, such stimuli can be delivered via one or more associated accessories before and/or during a blood collection procedure.

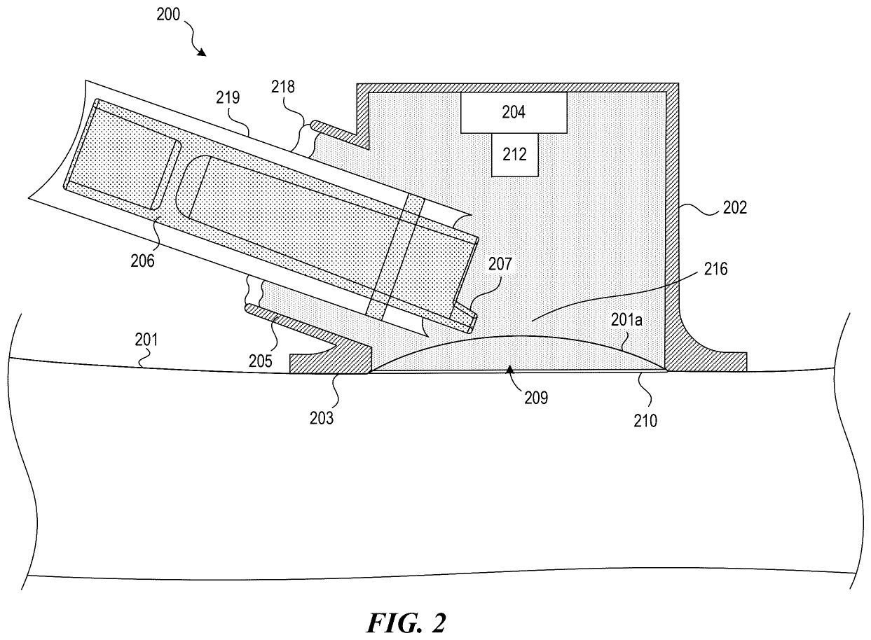

FIG. 2 is a partially schematic cross-sectional side view of a bodily fluid collection device 200 configured in accordance with an embodiment of the present technology. The device 200 can include a number of features generally similar to/identical to the features of the blood collection device 100 described with reference to FIGS. 1A-1C. Accordingly, like reference numbers refer to like components.

In the illustrated embodiment, the device 200 includes a housing 202, an actuator 204 (shown schematically), a skin-piercing assembly 212 (also shown schematically) located at least partially or completely within the housing 202, and an opening 209 through the housing 202. The opening 209 is formed in a bottom surface 210 of the housing 202 configured to be positioned against skin 201 of the patient. In the illustrated embodiment, the opening 209 is a large opening (e.g., 30 mm) extending completely across or substantially completely across the bottom surface 210 of the housing 202. In other embodiments, however, the opening 209 can have different dimensions and/or arrangements relative to the housing 202.

The skin-piercing assembly 212 includes at least one skin-piercing feature (e.g., a lancet, blade, or needle). The actuator 204 is operably coupled to the skin-piercing assembly 212 and movable within the housing 202 in both a deployment direction (toward the skin 201 of the patient) and a retraction direction (away from the skin 201). In operation, when the actuator 204 moves in the deployment direction, the skin-piercing feature is configured to pierce the skin 201 of the patient to create an incision from which bodily fluid can be withdrawn. Subsequent movement of the actuator 204 (in the retraction direction) creates a vacuum within the housing 202 that acts against the patient's skin 201, either directly or indirectly. Bodily fluid (e.g., blood) from the resulting incision is withdrawn into the housing 202 and, as explained in further detail below, collected into a collection reservoir 206 operably coupled to the housing 202.

In the illustrated embodiment, the device 200 includes a connector 205 angled relative to a base 203 of the housing 202 such that the collection reservoir 206 can extend at least partially through the connector 205 at and/or proximate the opening 209. In some embodiments, for example, a lip 207 of the collection reservoir 206 is configured to be positioned adjacent the incision formed in the skin 201 (via the skin-piercing assembly 212) such that blood collected through the opening 209 can flow directly from the incision into the collection reservoir 206. In some embodiments, the collection reservoir 206 is further coupled to fluidic channels that transfer blood from the incision point to the collection reservoir 206.

In some embodiments, as noted above, the device 200 includes a vacuum mechanism to facilitate collection of the bodily fluid from the patient. As vacuum pressure is applied, the skin 201 is drawn through the opening 209 into an interior space 216 of the housing 202 (e.g., creating a domed or curved skin interface region). In the present example, the opening 209 is sized such that it comprises substantially all of the bottom surface 210 of the housing 202, allowing for vacuum pressure to act on substantially all of the skin positioned under housing 202. As the skin 201a into the interior space 216, the lip 207 of the collection reservoir 206 contacts the drawn-in portion of skin 201a, allowing blood to flow from an incision site of the drawn-in portion of skin 201a into the collection reservoir 206. Increasing the surface area of the patient's skin 201a that can be drawn into the housing 202 during operation is expected to provide enhanced blood collection for the device 200.

In some embodiments, the device 200 can further include a flexible membrane 218 positioned to engage the collection reservoir 206. The flexible membrane 218 is configured to allows for the collection reservoir 206 to adjust position based on the position of the skin 201a drawn into the housing 202 and/or the amount of vacuum pressure. That is, the flexible membrane 218 allows the collection reservoir 206 to remain proximate to and/or in contact with the skin 201a regardless of whether the drawn-in portion of the skin 201a is only shallowly drawn into the interior space 216, or in procedures where the drawn-in portion of the skin 201a extends deeply into interior space 216. In some embodiments, the flexible membrane 218 is coupled to a moveable channel 219 configured to help position the collection reservoir 206 proximate to and/or in contact with the skin 201a. For example, the channel 219 can be configured such that an opening of the channel 219 is proximate and/or in contact with the skin 201a. The collection reservoir 206 can then be connected to the channel such that the lip 207 of the collection reservoir 206 is in contact with the skin 201a. As the portion of the skin 201a is drawn into the interior space 216, the flexible membrane 218 allows the channel 219 and collection reservoir 206 to move flexibly with the skin 201a. The flexible membrane 218 is an optional feature that may not be included in some embodiments.

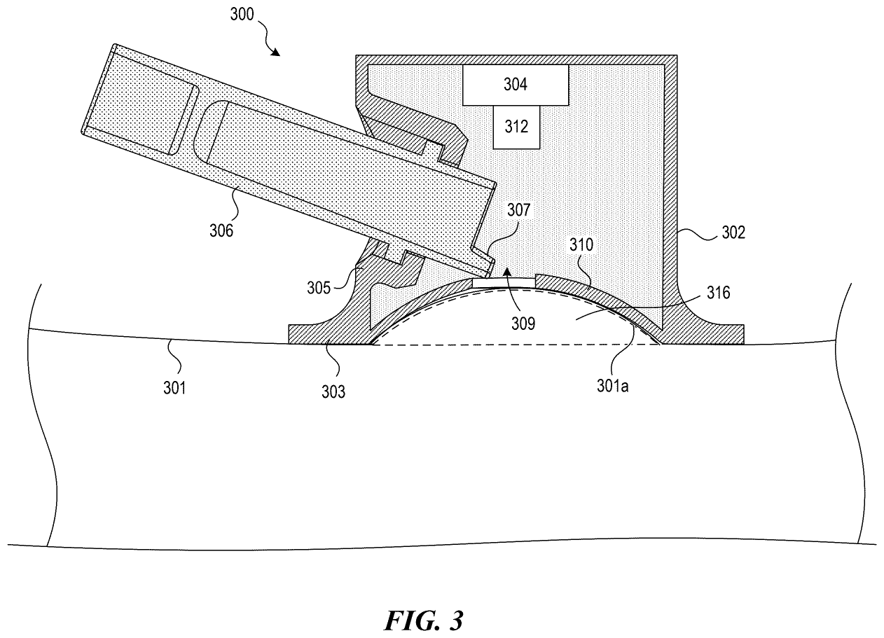

FIG. 3 is a partially schematic cross-sectional side view of a bodily fluid collection device 300 configured in accordance with another embodiment of the present technology. The device 300 can include a number of features generally similar to/identical to the features of the blood collection device 200 described with reference to FIG. 2. Accordingly, like reference numbers refer to like components.

The device 300 is principally distinct from the device 200 described above with reference to FIG. 2 in that the bottom surface 310 comprises a rigid dome-shaped region concave with respect to the housing 302. Furthermore, the bottom surface 310 comprises an opening 309 that does not substantially comprise all of the bottom surface 310. Put another way, the opening 309 allows a relatively smaller portion of skin 301 to be directly exposed to vacuum pressure than the opening 209 of the device 200. The rigid dome-shaped region formed by the bottom surface 310 at least partially defines an accommodating space 316 (represented by a dashed outline in FIG. 3) into which a portion of skin 301a may be drawn when exposed to vacuum pressure via the opening 309. In the illustrated embodiment, the dome-shaped bottom surface 310 is expected to allow for controlled doming/stretching of the drawn-in skin portion 301a and facilitate increased blood flow into local capillaries, while limiting bruising of the patient's skin.

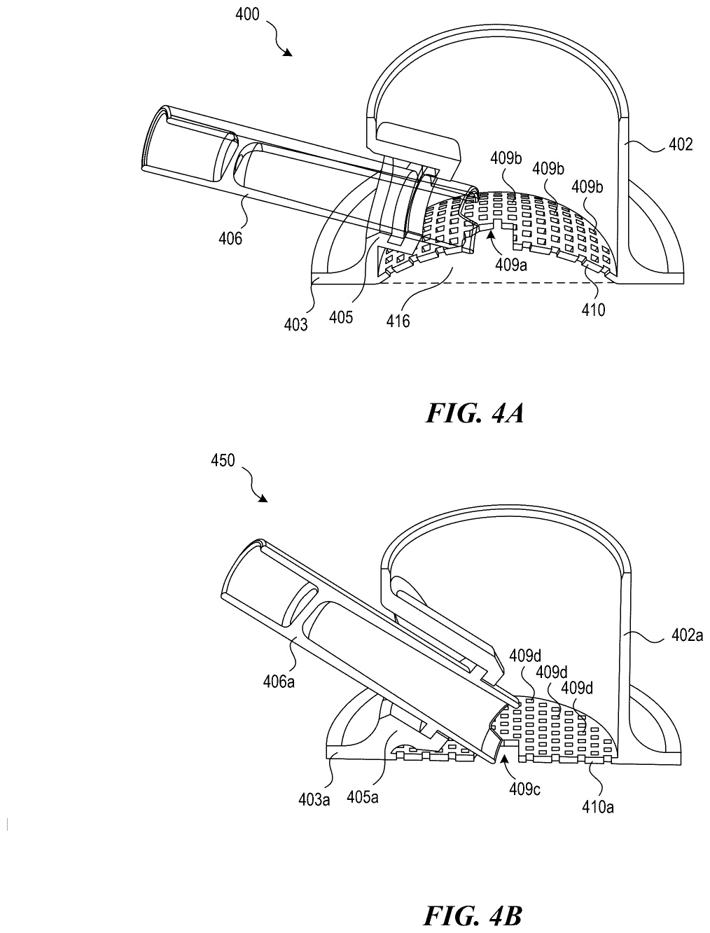

FIG. 4A is a partially schematic cross-sectional perspective view of a bodily fluid collection device 400 configured in accordance with embodiments of the present technology. The device 400 can include a number of features generally similar to/identical to the features of the blood collection devices 200 and 300 described with reference to FIGS. 2 and 3, respectively. Accordingly, like reference numbers refer to like components.

The device 400 is principally distinct from the device 300 in that a bottom surface 410 of the device 400 is comprised of a mesh feature having multiple openings, including a primary opening 409a and multiple secondary openings 409b arranged to form a mesh dome or mesh pattern. Similar to the bottom surface 310 of the device 300, the bottom surface 410 of the device 400 comprises a dome-shaped concave feature with respect to housing 402. The rigid dome-shaped arrangement of the bottom surface 410 at least partially defines an accommodating space 416 (represented by a dashed outline in FIG. 4A) into which a portion of skin may be drawn when exposed to vacuum pressure via openings 409a and 409b. In the present example, the accommodating space 416 extends throughout the concave area at least partially defined by the bottom surface 410. In some embodiments, the mesh bottom surface 410 is rigid or generally rigid. In other embodiments, however, the mesh bottom surface 410 is composed of a flexible material configured to stretch with the skin as the skin is drawn into the device during operation.

The multiple secondary openings 409b are configured to enhance the amount of skin exposed to vacuum pressure during operation of the device 400, which is expected to increase blood flow in the capillaries of the target skin region. The rigid dome structure is expected to control/inhibit the curvature of the skin drawn into the accommodating space 416, thus limiting bruising.

FIG. 4B is a partially schematic cross-sectional perspective view of a bodily fluid collection device 450 configured in accordance with another embodiment of the present technology. The device 450 is generally similar to/identical to the blood collection device 400 described with reference to FIG. 4A. The device 450 of FIG. 4B, however, differs from the device 400 of FIG. 4A in that a bottom surface 410a (comprised of a primary opening 409c and multiple secondary openings 409d) is configured to lie approximately flat and/or parallel to the skin when the device 450 is placed on the patient's skin (not shown). In such embodiments, the accommodating space is approximately equivalent to the space of the interface between the bottom surface 410a and the skin. In some embodiments, the accommodating space is omitted from the device 450. As vacuum pressure is applied to the target region of skin below the bottom surface 410a (e.g., via the primary and secondary openings 409c, 409d), the bottom surface 410a is configured to keep the skin approximately flat during operation of the device 450. Controlling/inhibiting skin curvature in this way is expected to mitigate bruising of the patient, while still facilitating enhanced local blood flow. In some embodiments, the mesh bottom surface 410a is shaped as part of the device 450. In other embodiments, however, the bottom surface 410a is provided as an insert/separate component that is applied to the device 450.

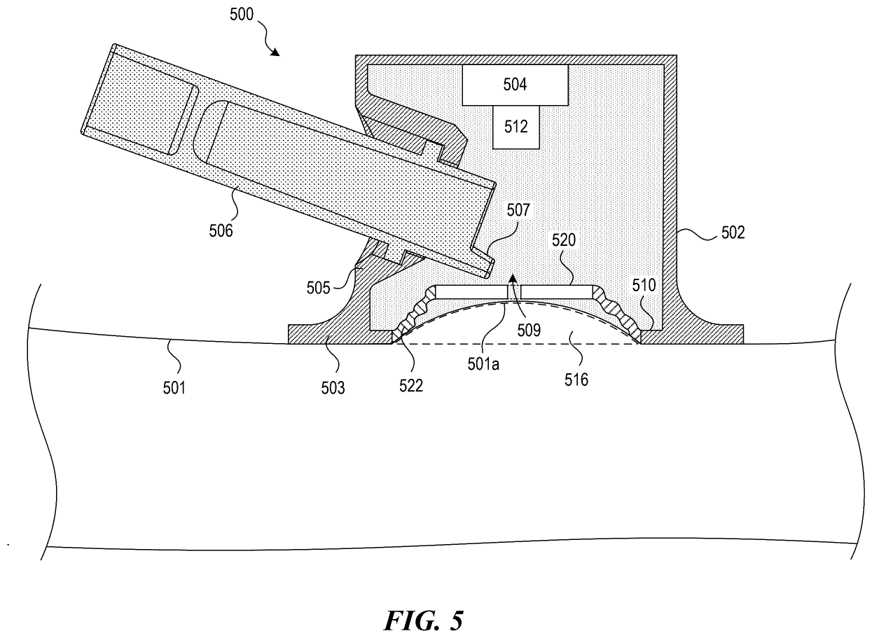

FIG. 5 is a partially schematic cross-sectional side view of a bodily fluid collection device 500 configured in accordance with further embodiments of the present technology. The device 500 can include a number of features generally similar to/identical to the features of the blood collection devices 200, 300, 400, and 450 described with reference to FIGS. 2, 3, 4A, and 4B, respectively. Accordingly, like reference numbers refer to like components.

In some embodiments, the bottom surface 510 includes a moveable platform 520 coupled to the base 503 via a flexible membrane 522. The moveable platform 520 is configured to remain in contact (e.g., raise and lower) with the patient's skin 501 as vacuum is applied, forcing a portion of the patient's skin 501a (e.g., the skin directly beneath the platform 520) to remain flat while allowing some doming/flexure around the edges of the platform 520. In some embodiments, the moveable platform 520 is configured to lie flat against the skin 501 (e.g., substantially planar). In some embodiments, the moveable platform 520 is contoured to form an accommodating space 516 for the skin (e.g., a concave dome structure). In some embodiments, the moveable platform 520 is rigid.

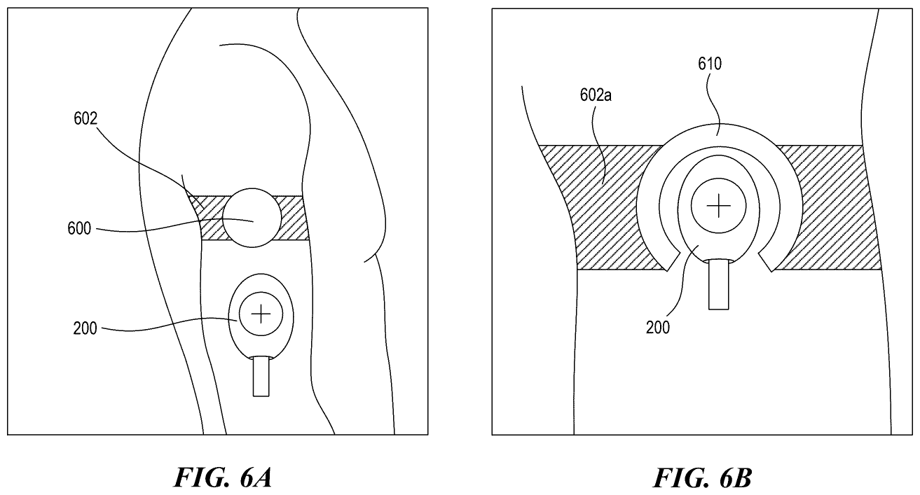

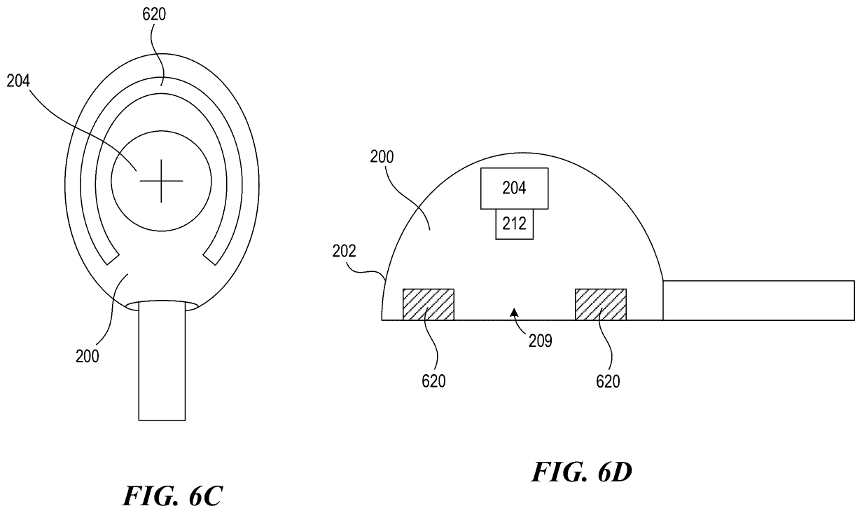

FIG. 6A is a perspective view of a bodily fluid collection device with a temperature/vibration accessory device 600 configured in accordance with embodiments of the present technology. The accessory device 600 can be configured to work with and/or be integrated in any of the bodily fluid collection devices disclosed throughout the present application and the references incorporated therein.

The accessory device 600 is a vibrating/heating device used to help promote increased blood collection. In some embodiments, for example, the accessory device 600 includes an electronic vibrating motor configured to generate physical pulses, motions, vibrations, agitations, and the like in skin at and/or adjacent to a bodily fluid collection device (e.g., device 200, shown throughout FIGS. 6A-6D). In some embodiments, the accessory device 600 includes a temperature element configured to deliver heat to an area of skin at or adjacent to device 200. In some embodiments, accessory device 600 includes both the vibrating motor and the temperature element. In the present example, the accessory device 600 is positioned above the bodily fluid collection device 200 (relative to the arm). The accessory device 600 can include an arm band 602 or other attachment mechanism (e.g., an adhesive) used to help position and maintain placement of the accessory device 600. In other embodiments, however, the accessory device 600 may be positioned differently relative to the bodily fluid collection device.

FIG. 6B is a perspective view of a bodily fluid collection device with a temperature/vibration accessory device 610 configured in accordance with another embodiment of the present technology. In the present example, the accessory device 610 is configured to be positioned adjacent to and around the outside of the housing of the bodily fluid collection device 200. For example, the accessory device 610 can be shaped in a semi-circle configuration with an open area within the semi-circle configured to accommodate the collection device 200. Such embodiments can aid a user (e.g., a patient) in achieving correct/effective placement of the accessory device 610 relative to the collection device 200. Similar to FIG. 6A, the accessory device 610 is configured to provide vibration and/or heat to an area adjacent (e.g., proximate) to the collection device 200.

FIG. 6C is a perspective view of a bodily fluid collection device with a temperature/vibration accessory device 620 configured in accordance with embodiments of the present technology. FIG. 6D is a partially cross-sectional side view of the bodily fluid collection device 620 with the temperature/vibration accessory device of FIG. 6C. Taking FIGS. 6C and 6D together, in the present example, the vibrating and/or heating mechanism of accessory device 620 is integrated with the bodily fluid collection device 200. For example, the vibrating/heating mechanisms of accessory device 620 can be built into the housing 202 of the collection device 200, such that the accessory device 620 forms a semi-circular shape around the actuator 204, skin-piercing assembly 212, and opening 209 of the collection device 200. The accessory device 620 is configured to provide vibration(s) and/or heat directly to and/or proximate to an incision area of a patient's skin.

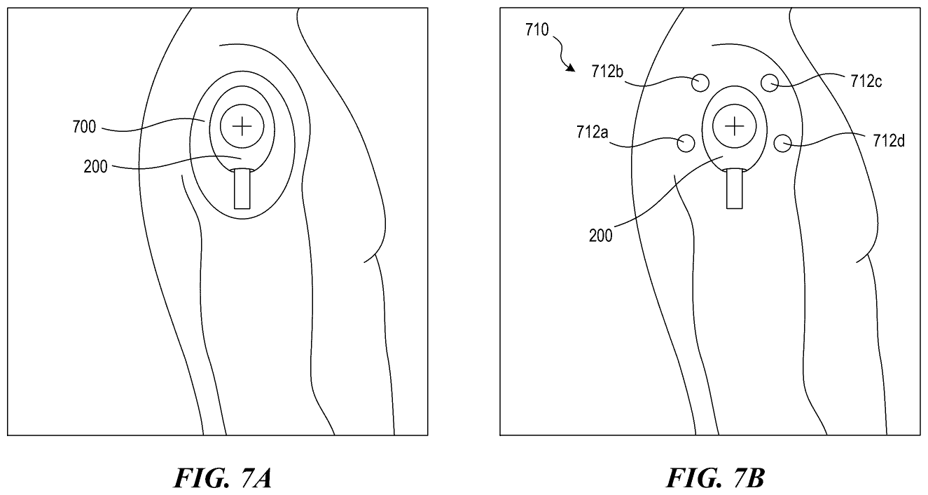

FIG. 7A is a perspective view of a bodily fluid collection device with a pressure accessory device 700 configured in accordance with further embodiments of the present technology. The accessory device 700 can be configured to work with and/or be integrated in any of the bodily fluid collection devices disclosed throughout the present application and the references incorporated therein.

The accessory device 700 is a pressure mechanism device configured to deliver positive or negative pressure to an area adjacent to (e.g., proximate) and/or including an incision site. For example, accessory device 700 can apply vacuum pressure (e.g., as a suction cup device) to an area encompassing bodily fluid collection device 200. In the present example, accessory device 700 is a suction cup device enclosing collection device 200. That is, the collection device 200 is contained within the suction cup accessory device 700. The suction force of the accessory device 700 provides a vacuum pressure on the area adjacent to and including the incision area of the collection device 200, increasing blood flow at the incision site. The accessory device 700 can include devices configured to provide compression, suction, and/or electrical pressure stimuli, among others.

FIG. 7B is a perspective view of a bodily fluid collection device with a pressure accessory device 710 configured in accordance with additional embodiments of the present technology. In the present example, accessory device 710 is comprised of multiple pressure elements 712a-d, each of which is positioned adjacent the collection device 200. For example, each of the pressure elements 712a-d can be comprised of suction cups configured to increase/enhance blood flow in the area local to the collection device 200.

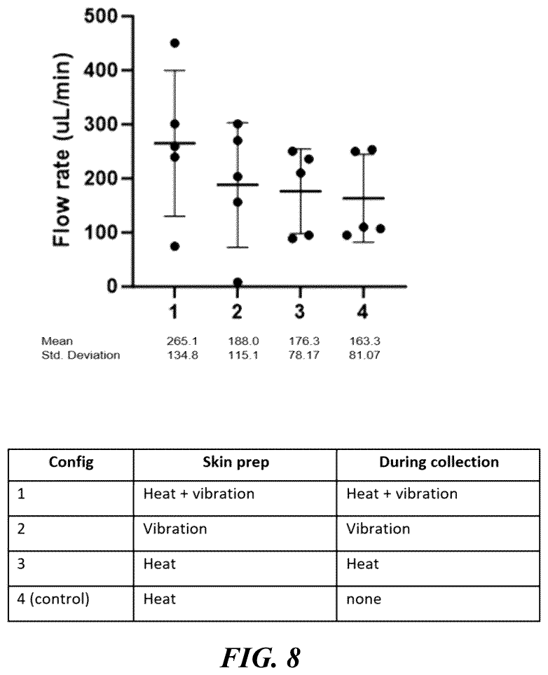

FIG. 8 is a display diagram and table based on data generated by the inventors in accordance with embodiments of the present technology. As illustrated, FIG. 8 indicates that applying heat and vibration during collection results in higher bodily fluid flow rates.

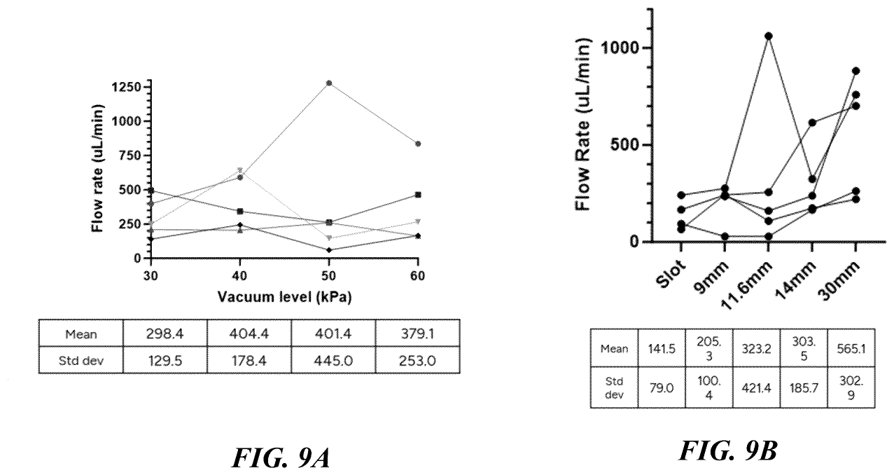

FIGS. 9A and 9B are display diagrams and tables based on data generated by the inventors in accordance with additional embodiments of the present technology. More specifically, FIG. 9A indicates an increase in bodily fluid flow rate up to 40 kilopascal (kPa), at which point flow rate levels off. FIG. 9B indicates there is a direct relationship between flow rate and the area of skin subjected to vacuum.

### C. Examples

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.
1. A device for withdrawing bodily fluid from a subject, the device comprising:
   a housing having a surface configured to be positioned against skin of the subject, the housing having an opening extending through the surface;
   a skin-piercing assembly positioned at least partially within the housing and having a skin-piercing feature configured to pierce the skin of the subject;
   an actuator assembly operably coupled to the skin-piercing assembly; and
   a collection cartridge operably coupled with the housing such that a portion of the collection cartridge is positioned proximate the opening;
   wherein the actuator is movable relative to the housing to deploy the skin-piercing feature through the opening and into the skin of the subject, and
   wherein—
      movement of the skin-piercing assembly away from the opening draws a vacuum within at least a portion of the housing, and the skin of the patient is configured to be drawn at least partially through the opening in the housing and assume a curved shape therein when the vacuum is drawn within the housing.
2. The device of example 1 wherein all or substantially all of the surface of the housing comprises the opening.
3. The device of either example 1 or example 2 wherein the opening has a diameter of about 30 mm.
4. The device of any of examples 1-3 wherein the surface is rigid, and wherein the surface is concave with respect to the housing.
5. The device any of examples 1-4, further comprising a mesh feature carried by the surface of the housing, and wherein the mesh feature includes the opening and a plurality of secondary openings.
6. The system of any of examples 1-5, further comprising a temperature accessory configured to apply heat and mechanical vibrations to a region of the skin proximate the skin contacted by the first surface.
7. The system of any of examples 1-6, further comprising a pressure accessory configured to apply pressure to a region of the skin proximate the skin contacted by the surface of the housing.
8. A device for withdrawing bodily fluid from a subject, comprising:
   a housing including a bottom surface configured to be positioned against skin of the subject and an opening extending through the bottom surface;
   a flexible membrane coupled to the housing and extending partially across the opening in the bottom surface;
   a platform coupled to the flexible membrane, wherein the flexible membrane and the platform are configured to be positioned in contact with the skin;
   a collection cartridge removably coupled with the housing;
   a skin-piercing assembly positioned at least partially within the housing and having a skin-piercing feature configured to pierce the skin; and
   an actuator operably coupled to the skin-piercing assembly and movable relative to the housing, wherein—
      movement of the actuator toward the opening deploys the skin-piercing feature through the opening and into the skin,
      movement of the actuator away from the opening draws a vacuum within at least a portion of the housing,
      the vacuum is configured to draw the flexible membrane, the platform, and at least a portion of the skin at least partially through the opening.
9. The device of example 8 wherein the platform is substantially planar.
10. The device of either example 8 or example 9 wherein the platform is contoured to form an accommodating space for receiving the skin of the subject.
11. The device of any of examples 8-10 wherein the platform has a concave dome shape.
12. The device of any of examples 8-11 wherein the platform is positioned at a center of the flexible membrane.
13. The device of any of examples 8-12 wherein the platform is rigid.
14. The device of any of examples 8-13 wherein the platform is configured to control skin curvature to mitigate bruising of the skin of the subject.
15. A bodily fluid collection system, comprising:
   a handheld housing including:
      a bottom surface in contact with skin of a patient and having an opening,
      a skin-piercing feature configured to form an incision in the skin through the opening,
      a flexible membrane coupled to the bottom surface and extending partially across the opening,
      a vacuum mechanism configured to apply vacuum pressure to the skin through the opening, and
      a platform positioned in contact with the skin and coupled to the bottom surface via the flexible membrane, wherein the vacuum pressure is configured to draw the flexible membrane, the platform, and at least a portion of the skin at least partially through the opening; and
   a collection reservoir fluidly coupled to the handheld housing and configured to collect bodily fluid flowing from the incision.
16. The bodily fluid collection system of example 15 wherein the collection reservoir includes a lip positioned adjacent to the incision.
17. The bodily fluid collection system of either example 15 or example 16 wherein the handheld housing further comprises a connector angled relative to the bottom surface, wherein the collection reservoir extends at least partially through the connector.
18. The bodily fluid collection system of any of examples 15-17 wherein the connector further comprises a moveable channel configured to help position the collection reservoir proximate to the skin.
19. The bodily fluid collection system of any of examples 15-18 wherein the handheld housing further comprises an actuator configured to deploy the skin-piercing feature.
20 The bodily fluid collection system of example 19 wherein at least a portion of the vacuum pressure is generated by movement of the actuator away from the bottom surface.
21. The bodily fluid collection system of any of examples 15-20 wherein the platform includes an opening configured to receive the skin-piercing feature and/or the bodily fluid.
22. The bodily fluid collection system of any of examples 15-21 wherein the platform includes an opening that is positioned coaxially with the opening of the bottom surface.
23. A method for collecting bodily fluid from a subject, comprising:
   positioning a collection device against skin of the subject, the collection device having:
      a housing including an opening and a platform positioned in the opening in contact with the skin;
   deploying a skin-piercing feature through the opening to create an incision in the skin;
   applying vacuum pressure through the opening to draw a portion of the skin, including the incision, and the platform into the housing; and
   collecting bodily fluid from the incision in a collection reservoir fluidly coupled to the housing.
24. The method of example 23 wherein deploying the skin-piercing feature and applying vacuum pressure comprises actuating an actuator of the collection device.
25. The method of either example 23 or example 24 wherein positioning the collection device comprises positioning a longitudinal axis of the collection reservoir parallel to a direction of gravity.
26. The method of any of examples 23-25, further comprising:
   removing the collection reservoir from the housing after the bodily fluid is collected.
27. The method of any of examples 23-26 wherein the housing further comprises a rigid bottom and a flexible membrane positioned between the rigid bottom and the platform.

### D. Conclusion

The above detailed description of embodiments of the present technology are not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps may be presented in a given order, in other embodiments, the steps may be performed in a different order. The various embodiments described herein may also be combined to provide further embodiments.

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

As used herein, the phrase and/or as in A and/or B refers to A alone, B alone, and A and B. Additionally, the term comprising is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. (canceled)

2. The device of claim 6 wherein the platform is substantially planar.

3. The device of claim 6 wherein the platform is contoured to form an accommodating space for receiving the skin of the subject.

4. The device of claim 6 wherein the platform has a concave dome shape.

5. A device for withdrawing bodily fluid from a subject, comprising:
a housing including a bottom surface configured to be positioned against skin of the subject and an opening extending through the bottom surface;
a flexible membrane coupled to the housing and extending partially across the opening in the bottom surface;
a platform coupled to the flexible membrane and positioned at a center of the flexible membrane, wherein the flexible membrane and the platform are configured to be positioned in contact with the skin;
a collection cartridge removably coupled with the housing;
a skin-piercing assembly positioned at least partially within the housing and having a skin-piercing feature configured to pierce the skin; and
an actuator operably coupled to the skin-piercing assembly and movable relative to the housing, wherein—
movement of the actuator toward the opening deploys the skin-piercing feature through the opening and into the skin,
movement of the actuator away from the opening draws a vacuum within at least a portion of the housing, and
the vacuum is configured to draw the flexible membrane, the platform, and at least a portion of the skin at least partially through the opening.

6. A device for withdrawing bodily fluid from a subject, comprising:
a housing including a bottom surface configured to be positioned against skin of the subject and an opening extending through the bottom surface;
a flexible membrane coupled to the housing and extending partially across the opening in the bottom surface;
a platform coupled to the flexible membrane, wherein the platform is rigid, and wherein the flexible membrane and the platform are configured to be positioned in contact with the skin;
a collection cartridge removably coupled with the housing;
a skin-piercing assembly positioned at least partially within the housing and having a skin-piercing feature configured to pierce the skin; and
an actuator operably coupled to the skin-piercing assembly and movable relative to the housing, wherein—
movement of the actuator toward the opening deploys the skin-piercing feature through the opening and into the skin,
movement of the actuator away from the opening draws a vacuum within at least a portion of the housing, and
the vacuum is configured to draw the flexible membrane, the platform, and at least a portion of the skin at least partially through the opening.

7. A device for withdrawing bodily fluid from a subject, comprising:
a housing including a bottom surface configured to be positioned against skin of the subject and an opening extending through the bottom surface;
a flexible membrane coupled to the housing and extending partially across the opening in the bottom surface;
a platform coupled to the flexible membrane, wherein the flexible membrane and the platform are configured to be positioned in contact with the skin;
a collection cartridge removably coupled with the housing;
a skin-piercing assembly positioned at least partially within the housing and having a skin-piercing feature configured to pierce the skin; and
an actuator operably coupled to the skin-piercing assembly and movable relative to the housing, wherein—
movement of the actuator toward the opening deploys the skin-piercing feature through the opening and into the skin,
movement of the actuator away from the opening draws a vacuum within at least a portion of the housing,
the vacuum is configured to draw the flexible membrane, the platform, and at least a portion of the skin at least partially through the opening, and
wherein the platform is configured to control a curvature of the skin to mitigate bruising of the skin of the subject.

8. (canceled)

9. The device of claim 6 wherein the collection cartridge includes a lip positioned adjacent to the skin.

10. The device of claim 6 wherein the housing further comprises a connector angled relative to the bottom surface, and wherein the collection reservoir extends at least partially through the connector.

11. A bodily fluid collection system, comprising:
a handheld housing including:
a bottom surface in contact with skin of a patient and having an opening,
a skin-piercing feature configured to form an incision in the skin through the opening,
a flexible membrane coupled to the bottom surface and extending partially across the opening,
a vacuum mechanism configured to apply vacuum pressure to the skin through the opening,
a platform positioned in contact with the skin and coupled to the bottom surface via the flexible membrane, wherein the vacuum pressure is configured to draw the flexible membrane, the platform, and at least a portion of the skin at least partially through the opening, and
a connector angled relative to the bottom surface, wherein the connector comprises a moveable channel; and
a collection reservoir configured to be fluidly coupled to the connector and to extend at least partially through the moveable channel, wherein the collection reservoir is configured to collect bodily fluid flowing from the incision, and wherein the moveable channel is configured to help position the collection reservoir proximate to the skin.

12. The bodily fluid collection system of claim 11 wherein the handheld housing further comprises an actuator configured to deploy the skin-piercing feature.

13. The bodily fluid collection system of claim 12 wherein at least a portion of the vacuum pressure is generated by movement of the actuator away from the bottom surface.

14. The device of claim 6 wherein the platform includes an opening configured to receive the skin-piercing feature and/or the bodily fluid.

15. The device of claim 6 wherein the platform includes an opening that is positioned coaxially with the opening through the bottom surface of the housing.

16-20. (canceled)
